(19) United States
(12) Patent Application Publication
(10) Pub. No.: US 2026/0283840 A1
(43) Pub. Date: Sep. 24, 2026

(54) PERSONAL THERMAL SKIN TREATMENT DEVICE

(71) Applicant: 9527-3165 Québec Inc., Mirabel (CA)

(72) Inventor: Ritchie DERAICHE, Mirabel (CA)

(21) Appl. No.: 19/571,028

(22) Filed: Mar. 18, 2026

Related U.S. Application Data
(60) Provisional application No. 63/774,455, filed on Mar. 19, 2025.

(51) Int. Cl. A61F 7/00

(52) U.S. Cl. CPC A61F 7/0053; A61F 7/007; A61F 7/0085; A61F 2007/0056; A61F 2007/0075

(57) ABSTRACT

A personal thermal skin treatment device comprises a basin configured to receive a liquid to submerge a body part of a user, a cooling module thermally coupled to the basin to cool the basin, a heating module to produce steam and a nozzle to direct the steam. The basin and/or at least part of the cooling module is thermally insulated from at least part of the heating module. All elements are at least partially housed in a housing of the personal thermal skin treatment device.

### CROSS-REFERENCE TO RELATED APPLICATIONS

The present patent application claims priority to provisional U.S. Application no. 63/774,455 filed Mar. 19, 2025 under 35 U.S.C. § 120 and is incorporated herein by reference in its entirety.

### FIELD OF THE INVENTION

The present description relates to a personal thermal skin treatment device. More specifically, the present description relates to a personal thermal skin treatment device or system that provides cold plunge treatments, and optionally, hot steam treatments.

### BACKGROUND OF THE INVENTION

Hot and cold treatments on the skin can provide many benefits to the user. Conventional cold facial treatments can include dipping the face into a bowl of cold water or ice water. However, this method requires time and preparation and can be messy. Similarly, heated facial treatments can include boiling water and directing the steam onto their face, for example with a towel over the head. However, this method also requires time and preparation and carries a significant risk of burning the skin, either the face or other body parts due to spills.

Accordingly, there is a need for a technology that overcomes at least some of the drawbacks of what is known in the field.

### SUMMARY OF THE INVENTION

It is an object of the present invention to provide a personal thermal skin treatment device or system that overcomes or mitigates one or more disadvantages of known thermal systems or at least provides a useful alternative.

In accordance with an embodiment of the present invention, there is provided a personal thermal skin treatment device or system comprising a housing, a basin for receiving a liquid to submerge a body part of a user, a cooling module thermally coupled to the basin and configured to cool the liquid in the basin, a heating module producing heat and thermal insulation. The thermal insulation is located between the heating module and the basin and/or between the heating module and the cooling module. The basin, the cooling module, the heating module and the thermal insulation are located at least partially within the housing.

Optionally, the thermal insulation is an insulation barrier creating compartments to at least partially contain the cooling module or the heating module; and/or a heat insulation panel positioned between the heating module and the basin; and/or a heat insulation panel positioned between the heating module and/or the cooling module.

Optionally, the basin may be removably insertable in the housing.

Optionally, the personal thermal skin treatment may further comprise a nozzle and a fluid tank for containing a liquid. The fluid tank may be connected to the nozzle.

Optionally, the personal thermal skin treatment may further comprise a light proximate the nozzle for lighting the steam directed by the nozzle.

Optionally, the heating module may be operable to produce steam by evaporating the liquid in the fluid tank using the heat. The nozzle may be operable to direct the steam produced by the heating module outside of the housing.

Optionally, the heating module may further comprise a heating element thermally coupled to the fluid tank. The heat may be at least partially produced by the heating element.

Optionally, the cooling module and the heating module may respectively be a cooling side and a heating side of a thermoelectric module. The cooling side may be being thermally coupled to a thermally conductive portion of the basin to cool the liquid in the basin. The heating side may be operable to produce the heat.

Optionally, the personal thermal skin treatment device may further comprise a spacer. The thermal insulation may be between the basin and the thermoelectric module to thermally insulate the basin from the heating side of the thermoelectric module. The spacer may extend through the thermal insulation and abut the basin to thermally couple the cooling side of the thermoelectric module to the basin through the thermal insulation.

Optionally, the personal thermal skin treatment device may further comprise an inner basin wall supporting the basin and an outer basin wall spaced apart from the inner basin wall. The thermal insulation may be between the inner basin wall and the outer basin wall.

Optionally, the thermal insulation is an air or vacuum gap between the inner basin wall and the outer basin wall.

Optionally, the inner basin wall and the outer basin wall may include an aperture to receive the spacer for thermally coupling the cooling side of the thermoelectric module to the basin through the thermal insulation.

Optionally, the aperture may comprise walls separating the spacer from the thermal insulation.

Optionally, the personal thermal skin treatment device may further comprise a heat sink thermally coupled to the heating side of the thermoelectric module.

Optionally, the personal thermal skin treatment device may further comprise an insulation layer between the cooling side of the thermoelectric module and the heat sink to thermally insulate the cooling side and the basin from the heat sink.

Optionally the personal thermal skin treatment device may further comprise a diffuser configured to disperse one or more essential oils. The diffuser may have a diffuser nozzle or a diffuser stone.

Optionally, the personal thermal skin treatment device may further comprise an ionizer in fluid communication with the basin. The ionizer may be operative to ionize the liquid in the basin.

In accordance with another embodiment of the invention, there is provided a personal thermal skin treatment device comprising a housing, a basin for receiving a liquid to submerge a body part of a user, a cooling module having a cooling side and a heating side, a heating module configured to produce steam, a nozzle to direct the steam produced by the heating module and thermal insulation. The cooling side is thermally coupled to the basin for cooling the liquid in the basin. The basin, the cooling module, the heating module, the thermal insulation and the nozzle are all located at least partially within the housing. The thermal insulation is in at least one of the following locations: between the basin and the heating side of the cooling module; between the basin and the heating module; between the cooling side of the cooling module and the heating side of the cooling module; and between the cooling side of the cooling module and the heating module.

Optionally, the cooling side and the heating side may be the respective cooling side and heating side of a thermoelectric module.

Optionally, the personal thermal skin treatment device may further comprise a light proximate the nozzle for lighting the steam directed by the nozzle.

### BRIEF DESCRIPTION OF DRAWINGS

These and other features of the present invention will become more apparent from the following description in which reference is made to the appended drawings wherein:

FIG. 1 is a perspective front view of a personal thermal skin treatment device according to one implementation showing a housing and a cover;

FIG. 2 is a perspective front view of the housing of the personal thermal skin treatment device shown in FIG. 1;

FIG. 3 is a cross-sectional front view of the personal thermal skin treatment device shown in FIG. 2 taken along lines 3 – 3;

FIG. 4 is a cross-sectional front view of the personal thermal skin treatment device shown in FIG. 2 taken along lines 4 – 4;

FIG. 5 is a cross-sectional side view of the personal thermal skin treatment device shown in FIG. 2 taken along lines 5 – 5;

FIG. 6 is a top view of the personal thermal skin treatment device shown in FIG. 1;

FIG. 7 is a cross-sectional back view of the personal thermal skin treatment device shown in FIG. 6 taken along lines 7 – 7;

FIG. 8 is a cross-sectional perspective back view of the personal thermal skin treatment device shown in FIG. 6 taken along lines 7 – 7;

FIG. 9a is an exploded cross-sectional back view of the personal thermal skin treatment device shown in FIG. 6 taken along lines 7 – 7;

FIG. 9b is a perspective view of the personal thermal skin treatment device shown in FIG. 1 with its cover open and the basin partially removed from the housing;

FIG. 10A is a top schematic view of a personal thermal skin treatment device according to another implementation showing a housing and a representation of a user utilizing the basin;

FIG. 10B is a back schematic view of the personal thermal skin treatment device shown in FIG. 10A;

FIG. 10C is a side partially transparent schematic view of the personal thermal skin treatment device shown in FIG. 10A;

FIG. 11A is a perspective front view of a personal thermal skin treatment device according to another implementation showing a housing and its cover;

FIG. 11B is a perspective front view of the housing of the personal thermal skin treatment device shown in FIG. 11A;

FIG. 11C is a perspective close-up view of the housing of the personal thermal skin treatment device shown in FIG. 11A;

FIG. 12 is a perspective front view of a personal thermal skin treatment device according to another implementation showing a housing and a cover hingedly coupled to the housing;

FIG. 13 is a perspective front view of a personal thermal skin treatment device according to another implementation showing a housing and a cover removably coupled to the housing;

FIG. 14 is a perspective close-up view of a personal thermal skin treatment device according to another implementation;

FIG. 15A is a top view of a personal thermal skin treatment device according to another implementation showing a housing;

FIG. 15B is a perspective top view of the personal thermal skin treatment device m shown in FIG. 15A showing a cover hingedly attached;

FIG. 16A is a top view of a personal thermal skin treatment device according to another implementation showing a housing and its cover;

FIG. 16B is a perspective top view of the personal thermal skin treatment device shown in FIG. 16A;

FIG. 17A is a top view of a personal thermal skin treatment device according to another implementation;

FIG. 17B is a perspective top view of the personal thermal skin treatment device shown in FIG. 17A;

FIG. 18A is a top view of a personal thermal skin treatment device according to another implementation showing a housing;

FIG. 18B is a perspective top view of the personal thermal skin treatment device shown in FIG. 18A with a cover coupled to the housing;

FIG. 19A is a perspective top view of a personal thermal skin treatment device according to another implementation showing a housing and a removable cover;

FIG. 19B is a perspective top view of the housing of the personal thermal skin treatment device shown in FIG. 20A;

FIG. 19C is a detail perspective view of the housing of the personal thermal skin treatment device shown in FIG. 20A showing a display panel;

FIG. 19D is a detail perspective view of the housing of the personal thermal skin treatment device shown in FIG. 20A showing a nozzle and a diffuser stone;

FIG. 20 is a detail perspective view of the top of a personal thermal skin treatment device according to another implementation, and

FIG. 21 is a perspective view of the personal thermal skin treatment device according to another implementation.

### DETAILED DESCRIPTION OF THE INVENTION

The present disclosure relates to a personal thermal skin treatment device, also known as a personal thermal skin treatment system, designed to provide cold plunge treatments, and optionally, hot steam treatments. In some implementations, the personal thermal skin treatment device can be used to provide facial treatments, where hot steam opens up pores, loosens dirt, and prepares the skin for deeper cleansing and where the cold plunge tightens pores and reduces inflammation, thus leaving the skin rejuvenated. Conveniently, the personal thermal skin treatment device can offer to its user both the cold plunge treatments and the optional hot steam treatments within a single device or apparatus.

The personal thermal skin treatment device can include a cooling module to cool a liquid in a basin configured to receive a body part of the user, such as the face. The cooling module can include any cooling device, such as a thermoelectric module (which can also be referred to as a Peltier device), circulating a coolant, or other known cooling systems. In the exemplary implementations, a thermoelectric module having a cooling side is utilized. The basin is thermally coupled to the cooling side of the thermoelectric module to cool the liquid in the basin. The basin can be made, at least in part, of material configured to maintain a temperature for a period of time (such as a low thermal conductivity material) and/or include a temperature-control system configured to control the cooling module to keep the liquid in the basin at a desired temperature. In some implementations, the basin is removable to facilitate maintenance, cleaning, and/or to fill/empty the basin.

The personal thermal skin treatment device can include a heating module that heats a liquid to produce steam. In some implementations, the heating module produces steam that can be discharged through at least one nozzle to provide a steam treatment to the body part of the user. The at least one nozzle can be in the housing of the personal thermal skin treatment device or can be in the basin. For example, the heating module can be a steam-generating device configured to heat a liquid, such as water, and generate steam that is discharged from the personal thermal skin treatment device via the nozzle. In some implementations, the steam-generating device can include resistive heaters which generate steam when water is released onto their surface. The steam-generating device may be of the type operable to generate nano-ionic steam having smaller sized steam molecule (nano sized) than normal steam molecules to better penetrate into the skin of the user.

In some implementations, the heating module can heat the liquid in the basin to provide a heat treatment to the body part of the user. The heating module can include the heating side of the thermoelectric module used as the cooling module (i.e., the thermoelectric module can act as the cooling module and, at least in part, as the heating module). In some implementations, the cooling module and the heating module are thermally insulated from each other. As a non-limiting example, the thermoelectric module is a Peltier device (also known as a Peltier heat pump, a solid-state refrigerator, a thermoelectric cooler (TEC) and occasionally a thermoelectric battery). The primary advantages of a Peltier cooler compared to a vapor-compression refrigerator are its lack of moving parts or circulating liquid, very long life, invulnerability to leaks, small size, and flexible shape. Such Peltier device are well known in the art. With reference to the Figures below, the device is relatively compact, user-friendly, and aesthetically pleasing while remaining functional. The compact configuration enables the device to be installed on a bathroom counter, for example, and further allows the device to be stored in standard drawers and/or cabinets.

In some implementations, the personal thermal skin treatment device includes an integrated essential oil diffuser, which allows the device to remain functional even when the thermal systems (i.e. the hot steam or cold plunge features) are not being utilized.

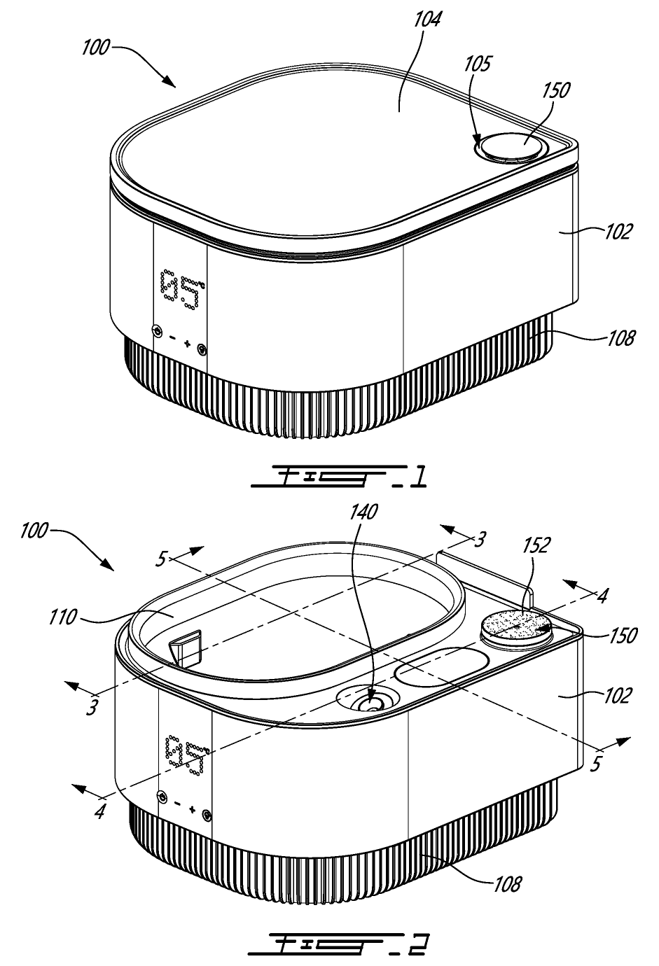

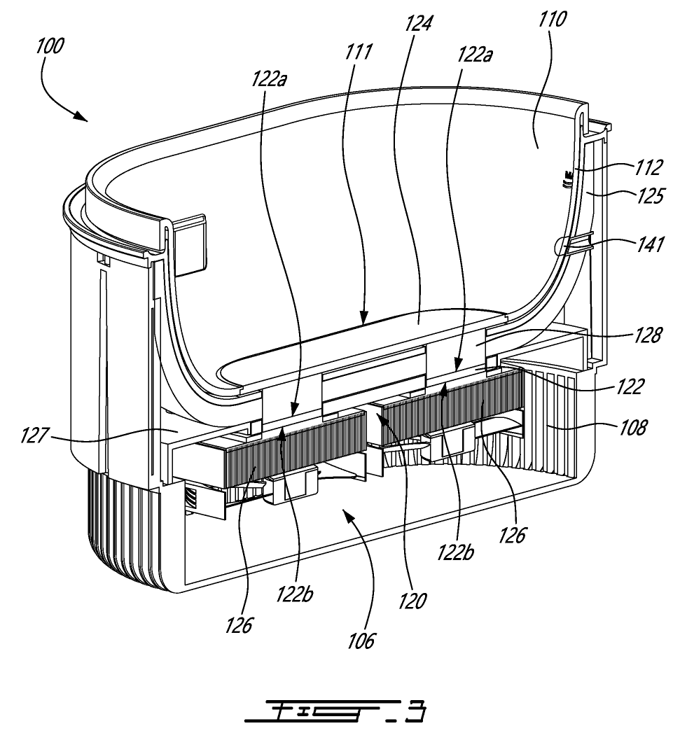

Referring now concurrently to FIGS. 1-5, there is provided a personal thermal skin treatment device 100 according to one implementation. The personal thermal skin treatment device includes a housing 102, a basin 110 configured to receive a liquid to submerge a body part of the user, a cooling module 120 configured to cool the liquid in the basin, a heating module 130 configured to heat a liquid, a nozzle 140 configured to expel or discharge steam, and a diffuser 150 configured to disperse one or more essential oils into the air. The personal thermal skin treatment device can be battery-powered (i.e., include a battery, such as in the housing 102) and/or include a power cord extending from the housing 102 to power the cooling module 120 and/or heating module.

The housing 102 can include a closeable lid or cover 104 that is configured to cover at least a portion of the housing 102 when not in use. For example, the cover 104 can protect the basin 110 when not in use. In the exemplary implementation, the cover 104 includes an aperture 105 configured to align with the nozzle 140 and the diffuser 150. In the exemplary implementation, the cover 104 is completely removable and covers the basin 110 and upper surface of the housing 102, while the aperture 105 leaves the diffuser 150 and nozzle 140 uncovered, such that they can continuously be used while the basin is covered. In some implementations, the cover 104 can be coupled to the housing 102 with a hinge.

In some implementations, the housing 102 includes a display panel (not shown) to control various aspects of the personal thermal skin treatment device 100. For example, the display panel can be coupled to a control system configured to activate/deactivate the cooling module and/or heating module, set and adjust a temperature or a range of temperatures of the liquid the cooling module and/or heating module are configured to cool or heat to, respectively, set or adjust a timer to maintain the liquid in the basin 110 or being expelled from the nozzle 140 at a specific temperature, etc. In some implementations, the personal thermal skin treatment device 100 can include a temperature control system having a sensor (not shown) in or near the basin 110 that measures a temperature of the liquid in the basin 110 and sends the measured temperature to the temperature control system. The temperature control system is configured to activate and deactivate the thermoelectric module based on the measured temperature from the sensor to maintain the temperature of the liquid in the basin 110 to the temperature set by the user or within a temperature range set by the user. In the present application, the thermoelectric module is selected to be able to cool water contained in the basin 110 to within 0º C and 10º C. The temperature control system may control water temperature as a function of the type of skin of the user.

The basin 110 can be shaped and configured to receive a liquid in a suitable volume where a body part of a user can be submerged. In the exemplary implementation, the basin 110 is oval shaped, having a length of about 30 cm and configured to receive, among other potential body parts, a face of the user. The basin 110 can hold any suitable volume of liquid, such as 500 ml, 1 L, 1.5 L, 2 L, or more. In the exemplary implementation, the basin 110 is configured to hold up to 1.5 L of liquid.

The basin 110 can be made of any suitable material impervious to liquids. Consideration to the thermal conductivity of the material of the basin 110 can be given depending on the configuration of the cooling module. For example, in the exemplary implementation as best shown in FIGS. 3, the basin 110 includes apertures 111 each configured to receive a portion of the cooling module 120 (in this implementation, a plate 124 of the cooling module 120) and thereby directly cool the liquid in the basin 110. Therefore, it can be advantageous for the basin 110 to be made of a low thermal conductivity material (i.e. with high insulation properties) to prolong the time the water in the basin 110 remains at the desired temperature. However, it is also contemplated that the cooling module 120 can abut a bottom and/or side of the basin 110 (i.e., the basin 110 does not include any apertures 111 and is in direct or indirect contact with the cooling module 120). Therefore, it can be advantageous for the entire basin 110 or at least the portions abutting the cooling module 120, to be made of a high thermal conductivity material that facilitates a thermal transfer between the cooling module 120 and the liquid in the basin 110.

The cooling module 120 includes any cooling device, such as circulating a coolant or refrigerant or other known cooling systems. In the exemplary implementation, with specific reference to FIG. 3, the cooling module 120 includes two thermoelectric modules 122, each having a cooling side 122a and a heating side 122b. However, it is contemplated that the cooling module 120 includes one or three or more thermoelectric modules 122.

Each cooling side 122a of the thermoelectric modules 122 are configured to abut, directly or indirectly, the basin 110 to thereby cool the liquid in the basin 110. In the exemplary implementation, each cooling side 122a directly abuts a spacer 128, which directly abuts a plate 124 that is coupled in each of the apertures 111 in the basin 110. However, as noted above, it is also contemplated that the cooling side 122a directly abuts an underside and/or side of the basin 110. Alternatively, the spacer 128 or the plate 124 could directly abut the underside or side of the basin 110 or the plate 124 could be integrally formed with the basin 110.

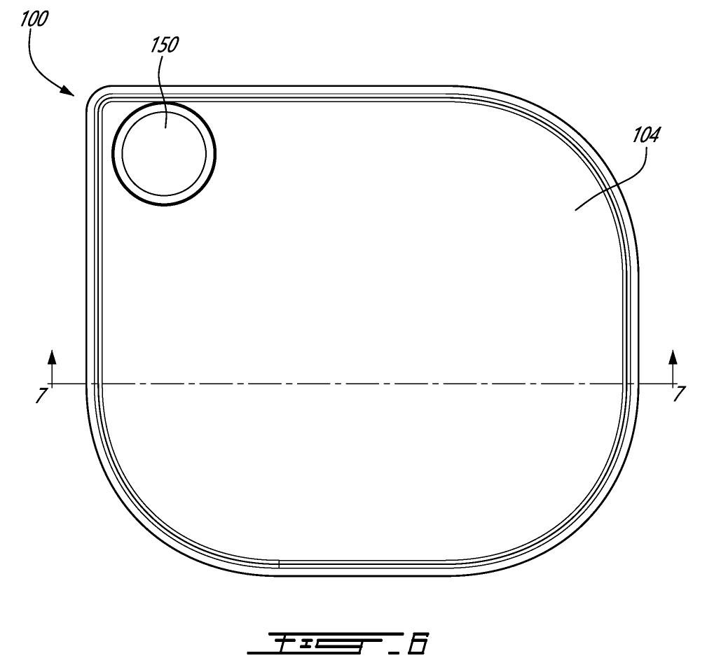

Each heating side 122b of the thermoelectric modules 122 can abut, directly or indirectly, a heat sink 126 that is configured to dissipate the heat generated by the cooling module 120 and expelled on the heating side 122b. In some implementations, the cooling module 120 can include a fan (not shown) to help dissipate the heat generated by the cooling module 120. As best shown in FIG. 7, in the exemplary implementation, the bottom side of the housing 102 includes a cavity 106 and a vent 108 configured to facilitate an airflow F (with or without the use of a fan) to further dissipate and expel the heat from the personal thermal skin treatment device 100.

In some implementations, the basin 110 can be provided with insulation 125 between the basin 110 and the heating side 122b of the cooling module 120 and/or the heat sinks 126 to insulate the basin 110 from the heat being dissipated in the cavity 106. In some implementations, the basin 110 can be provided with a second insulation layer 127 between the insulation 125 and the heat sinks 126. The insulation 125 or insulation layer 127 can be any suitable insulation, such as an air gap, radiant barriers, reflective insulation, phenolic foams, vermiculite, perlite, vacuum space, etc. Consideration to the overall weight of the personal thermal skin treatment device 100 can be given when determining a type of insulation 125 and/or insulation layer 127.

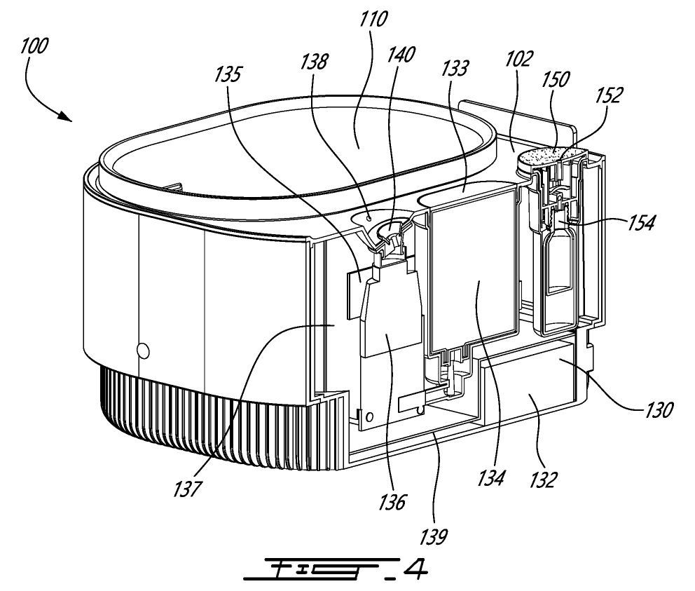

With specific reference to FIGS. 4 and 5, in the exemplary implementation, the heating module 130 includes a heating element 132 abutting or in fluid communication with a fluid tank 134 configured to hold a heatable liquid, and a steam chamber 136 in fluid communication with the fluid tank 134 and configured to expel the steam generated by the heating element 132 out the nozzle 140 in the housing 102. When actuated, the heating element 132 heats or boils the heatable fluid in the fluid tank 134 to generate steam in the steam tank 136. When a steam release is actuated (for example, by the control panel), the steam is expelled from the steam chamber 136 through the nozzle 140 and towards the user (for example, towards the user’s face for a steam facial treatment).

It is contemplated that the heating module 130 can include any style of steam generator, such as a heating element 132 directly coupled to the steam chamber 136, where the nozzle 140 is removable such that the user can fill the steam chamber 136 with the heatable fluid and the heating element 132 generates the steam directly in the steam chamber 136. In some implementations, the heating module 130 can utilize, at least in part, the heat from the heating side 122b of the thermoelectric module 122 of the cooling module 120. The steam generator may be of the type capable of generating nano-ionic steam.

In the exemplary implementation, the fluid tank 134 can include a removable cover 133 that the user can remove to fill the fluid tank 134 with the heatable liquid. The heatable liquid can include any liquid that can be heated to produce a steam vapour, such as water, water mixed with other components (such as essential oils), etc.

In some implementation, a light 138 is placed within or proximate to the nozzle 140 to illuminate the generated jet of steam. The light 13 may be an LED light. Its colour may be selected by the user, be determined as a function of predetermined parameters, or be controlled to vary with time. The light 138 may be placed on the housing 102 close to the nozzle 140 or within the nozzle 140 itself. The light 138 can be configured to illuminate the jet of steam from underneath.

In some implementations, additional insulation can be provided between the cooling module 120 and the heating module 130. In the exemplary implementation, as best shown in FIGS. 4 and 5, the personal thermal skin treatment device 100 includes a heat insulation panel 135, a first insulation barrier 137, and a second insulation barrier 139. The heat insulation panel 135 is coupled directly adjacent to the steam chamber 136 to thermally insulate the steam chamber 136 from the basin 110. The first insulation barrier 137 and the second insulation barrier 139 can form compartments in the housing 102 and provide additional thermic insulation between the coiling module 120 and/or basin 110 and the heating element 132 and/or other steam components (water tank 134, steam chamber 136, and/or at least one nozzle 140). In the exemplary implementation, the first insulation barrier 137 is a vertical barrier that provides separation between the water tank 134, steam chamber 136, and at least one nozzle 140, and the cooling module 120 and basin 110. The second insulation barrier 139 is a horizontal barrier that provides separation between the heating element 132 and the cooling side 122a of the thermoelectric module 122 and the other components of the heating module (water tank 134, steam chamber 136, and/or at least one nozzle 140).

In the exemplary implementation, the basin 110 is thermally insulated from the heating side 122b of the thermoelectric module 122 via insulation 125, insulation layer 127, base 118, and second insulation barrier 139. The basin 110 is also thermally insulated from the heating module 130 via insulation 125, first insulation barrier 137, and heat insulation panel 135.

The cooling module 120 is thermally insulated from the heating module 130 via first insulation barrier 137, second insulation barrier 139, and heat insulation panel 135. More specifically, the cooling side 122a of the thermoelectric module 122 is thermally insulated from the heating module 130 via insulation layer 127, base 118, first insulation barrier 137, second insulation barrier 139, and heat insulation panel 135.

The nozzle 140 can include any suitable nozzle configured to direct a jet of steam towards a body part of the user, such as the face. In some implementations, the nozzle or nozzles 140 are convergent nozzles configured to produce a fine mist of steam without causing excessive heat on the user’s body part. In some implementations, the nozzle or nozzles 140 can be adjustable to vary the steam intensity such that the user can customize the steam output. In some implementations, the nozzle or nozzles 140 are adjustable to vary the angle the steam is dispersed at, for example, rotating nozzles can be utilized to allow for maneuverability and targeted steam application across the body part.

A window 141 may be provided in the inner basin wall 112 to let blue light flow in the basin 110 to provide a colder aspect to the liquid contained in the basin 110. Alternatively to the window 141, a LED light could be directly installed in the inner basin wall 112 to provide this blue light.

In some implementations, the personal thermal skin treatment device can include a diffuser 150 configured to emit an aroma or mist of essential oils. The diffuser 150 may emit the aroma actively by forced convection (for example, with a fan) or passively by natural convection. As best shown in FIG. 4, in the exemplary implementation, the diffuser 150 includes an opening 152 in the housing 102 and a conduit 154 between the heating element 132 and the opening 152. The diffuser 150 further includes a diffusing stone (not shown) that is removably coupled to the housing above the opening 152. The user can add a fragrant liquid, such as oil or essential oils, to the diffusing stone. Warm air heated by the dissipated heat in the cavity 106 is circulated, using fans, from the cavity 106 through the conduit 154 and then through the diffusing stone , thereby dispersing a scented mist in the ambient environment from the oils contained in the diffusing stone . The diffusing stone may be made from a porous stone such as a volcanic rock.

In some implementations, the diffuser 150 can have a heating component that is separate from the heating module 130 (i.e., a separating heating element to heat the oils that is separate from the heating element 132 of the heating module 130). In some implementations, the heating component of the diffuser 150 can be, at least in part, the heat being dissipated from the heating side 122b of the thermoelectric module 122 of the cooling module 120.

The diffusing stone can be made of a porous material that allows the scent to dissipate into the room the personal thermal skin treatment device 100 is in. For example, the diffusing stone can be made of pumice stone or other porous rock to allow the heated oil to seep into the diffusing stone and diffuse into the air.

In some implementations, the diffuser 150 or the diffusing stone can be removed to clean the diffuser or the diffusing stone and/or to add the oil to the conduit 154 to be directly or indirectly heated by the heating element 132 (or other heating component). In other implementations, the diffusing stone can be permanently secured above the opening 152 and is configured to receive the oil directly, where a heated fluid moving from the heating element 132 (or other heating component) through the conduit 154 and out the opening, heats the oil in the diffusing stone to dissipate the oil through the air. For example, the heated fluid moving through the conduit 154 can be heated air or heated steam from the heating module 130. When the diffuser 150 utilizes steam, the conduit 154 can be indirectly coupled to the heating element 132 via the steam chamber 136.

Other implementations of diffusers 150 are also contemplated, such as the diffuser 150 utilizing a nozzle (not shown) to diffuse the heated oil from the conduit 154, as opposed to the diffusing stone, or the housing 102 can include a small bowl to hold the oil and the heating element 132 (or other heating component) directly or indirectly heats the small bowl to dissipate the oil in the air.

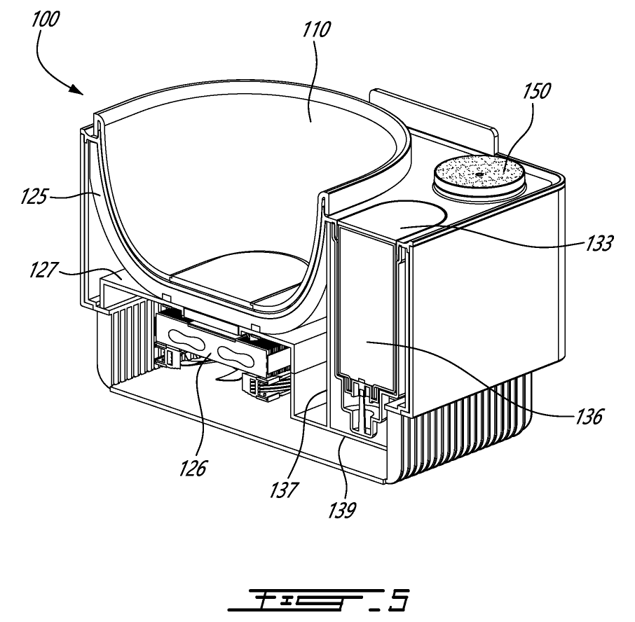

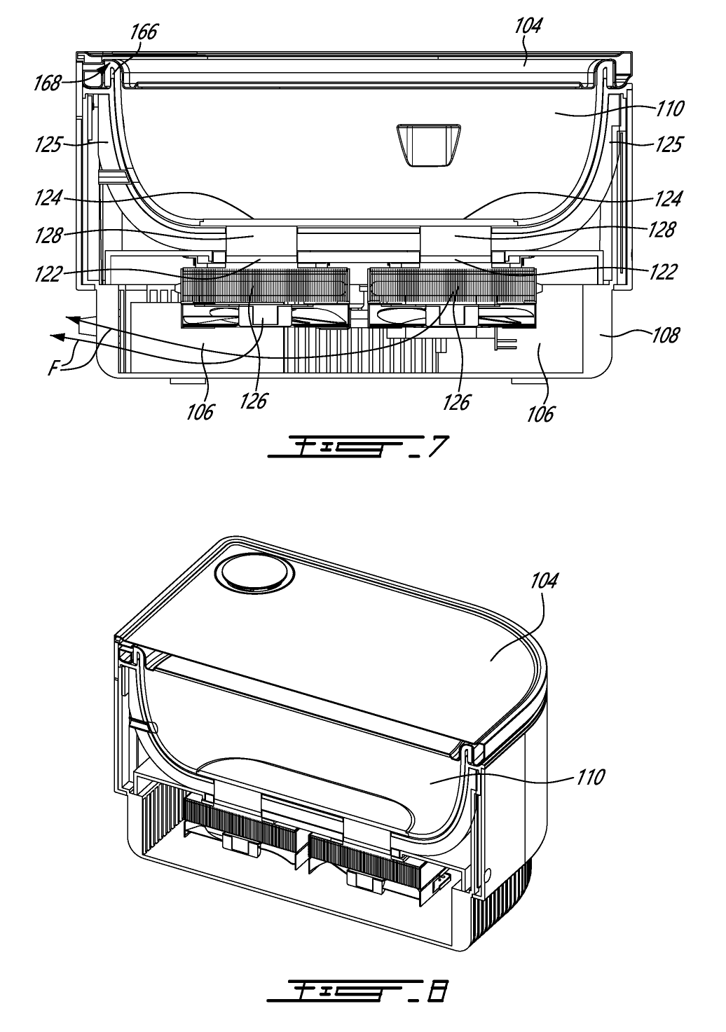

Referring now to FIGS. 6-9, a schematic of the personal thermal skin treatment device 100 is shown. As best shown in FIGS. 9a and 9b, the basin 110 is removably coupled to the housing 102 to facilitate cleaning, maintenance, filling of the liquid, etc. The basin 110 includes a flange 162 and a lip 164 creating a groove 163 that is configured to be received on a protrusion 166 on the housing 102. The basin 110 can be removably coupled to, or removably and partially inserted in, the housing 102 by placing the protrusion 166 in the groove 163 (for example, by resting the flange 162 on a top surface of the protrusion 166 and/or resting the lip 164 on a top surface of the housing 102). In this implementation, the cover 104 includes a groove 168 configured to receive the flange 162 and lip 164 on the basin 110 when the basin 110 is coupled to the housing 102, and the cover 104 is coupled to the basin 110 and housing 102.

The housing 102 includes an inner basin wall 112 that abuts the basin 110 when the basin 110 is coupled to the housing and an outer basin wall 114 that is spaced apart from the inner basin wall 112. As is best shown in FIGS. 7 and 8, this creates an air gap that is the insulation 125 between the outer basin wall 114 and the inner basin wall 112 (and thus between the cooling module 120 and the basin 110). In the exemplary implementation, the inner basin wall 112 extends above a top surface of the housing 102 to form the protrusion 166.

Referring back to FIG. 9, in this implementation, the basin 110 includes apertures 111 that are configured to receive the plates 124 of the cooling module 120. However, it is understood that the apertures 111 and the basin 110 are coupled in a manner that provides a fluid seal between the basin 110 and the inner basin wall 122 or the insulation 125 between the inner basin wall 112 and the outer basin wall 114 (i.e., fluid does not leak through the apertures 111). In some implementations, the plates 124 can be integrally formed with the basin 110 or the plates 124 act as plugs in the apertures 111 to prevent fluid communication between the basin 110 and the inner basin wall 112. In other implementations, the basin 110 can be formed of the same material as the plates 124 to allow the cooling side 122a of the thermoelectric module 122 to cool the liquid in the basin 110 (i.e., the entire basin 110 acts as a plate 124). For example, the cooling side of 122a of multiple thermoelectric modules 122 can abut, directly or indirectly, multiple areas around the bottom and/or sides of the basin 110.

Similarly, the inner basin wall 112 includes apertures 113 and the outer basin wall 114 includes apertures 115. The apertures 113 and apertures 115 are aligned and configured to receive the spacers 128 therethrough, which can allow the spacers 128 to directly abut the plates 124 in the basin 110 and/or directly abut the basin 110. In the exemplary implementation, the apertures 113 and apertures 115 provide a channel for the spacers 128 that allow the spacers 128 to directly abut the plates 124. The spacers 128 are configured to thermally transfer the cool energy from the cooling side 122a through the insulation 125 to the plates 124. However, it is also contemplated that the cooling side 122a of the thermoelectric module directly abuts the plates 124 and/or the basin 110. In the exemplary implementation, the apertures 115 in the outer basin wall 114 includes walls 116 that at least partially form a conduit between the inner basin wall 112 and the outer basin wall 114 to receive the spacers 128. In some implementations, the walls 116 can be insulated to prevent or reduce energy dissipation out the sides of the spacers 128 and concentration the cold energy channeled from the cooling side 122a of the thermoelectric module 120 towards the basin 110.

Similarly, the second insulation layer 127 includes apertures 117, each configured to receive the thermoelectric module 122. The apertures 117 allows the cooling side 122a to abut the spacers 128 and the heating side 122b to abut a heat sink 126, while insulating the sides of the thermoelectric module 122 to prevent a heating of the cooling side 122 and vice versa.

In some implementations, the personal thermal skin treatment device 100 can include a base 118 that provides support for the basin 110, the inner basin wall 112, and the outer basin wall 114. The base 118 can include apertures 119 aligned with the apertures 115 and apertures 117 to allow thermal communication or thermal transfer between the spacers 128, the thermoelectric module 122, and the heat sink 126.

In some implementations, the personal thermal skin treatment device 100 can include an ionizer (not shown) configured to ionize the liquid in the basin 110, the heatable liquid in the fluid tank 134, and/or the steam in the steam chamber 136. The ionizer can be in fluid communication with the basin 110, fluid tank 134, and/or steam chamber 136. For example, an ionizer can be contained in the housing 102 and the basin 110 can include an outlet let to allow the liquid to flow to the ionizer and an inlet to recirculate the ionized liquid back to the basin 110. Similarly, the personal thermal skin treatment device 100 can include sterilizing or purification devices to sterilize or purify the liquid in the basin 110 and/or the heatable fluid in the fluid tank 134, such as UV radiation, ozone treatments, etc.

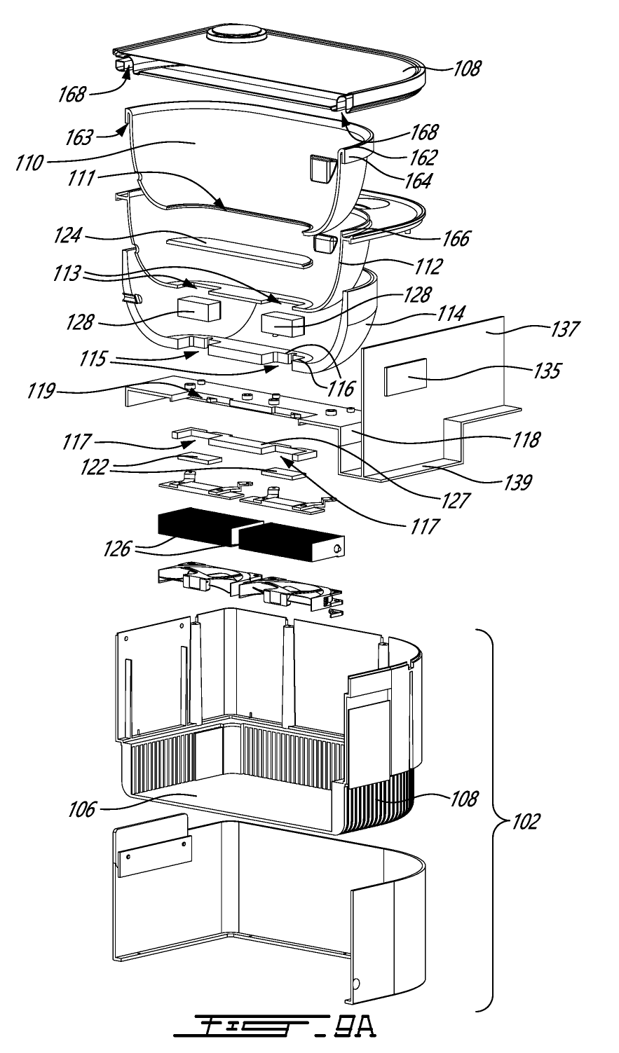

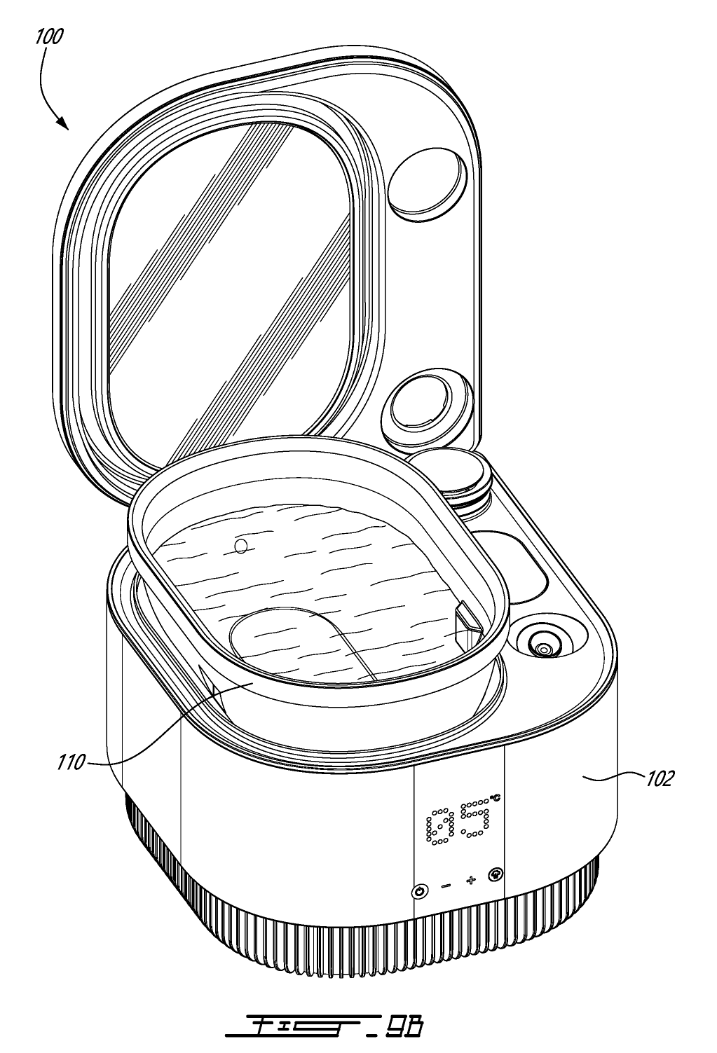

Referring now to FIGS. 10A-10C, a personal thermal skin treatment device 200 according to another implementation is shown in use with a representative of a user 1. The personal thermal skin treatment device 200 includes a housing 202 supporting a basin 210 on an external side thereof and containing a cooling module 220 and a heating module 230 having a steam chamber 236 on an internal side thereof. The cooling module 220 is configured to cool a liquid in the basin 210, allowing the user 1 to soak their face in a cold plunge treatment. The external side of the housing 202 also includes a nozzle 240 to facilitate a steam facial treatment for the user 1 (the user 1 is not shown in position over the nozzle 240), and a control panel 270 configured to allow the user 1 to control all functions of the personal thermal skin treatment device 400 and namely to actuate the cooling module 220 and/or heating module 230 (i.e., turn on and off), set a desired temperature or temperature range for the cooling module 220 and/or heating module 230, set or adjust a timer to maintain the liquid in the basin 210 at the desired temperature or expel steam from the nozzle 240 for a predetermined time. The housing 202 further contains a vent 208 on a bottom side thereof to assist with the dissipation of undesirable hot or warm air generated by the cooling module 220 and/or heating module 230.

It is understood that any size, shape, or configuration of the housing 202 can be used. Consideration to the body part the basin 210 is configured to receive should be given when determining a size of the basin 210 and housing 202. In the exemplary implementation, the basin 210 is configured to receive a face of a user 1 for facial treatments. As such, the basin 210 is sized and shaped to receive a human face. The upper part of the housing 202, which contains the basin 210, cooling module 220, and at least part of the heating module 230, has a depth of about 11 cm (109.91 mm), a width of about 32 cm (319 mm), and a length of about 32 cm (319 mm). The bottom part of the housing 202 containing the vent 208, and optionally one or more heat sinks and/or fans (not shown) has a depth of about 6 cm (60.6 mm), thus the personal thermal skin treatment device 200 has an overall depth of about 17 cm (170.51 mm). However, it is understood that any size and configuration can be used. For example, when a vent 208 is used, consideration should be given to allow for sufficient airflow out the vents 208 when determining the depth, width, and/or length of the bottom part. In the exemplary implementation, the bottom part has a smaller width and length than the upper part.

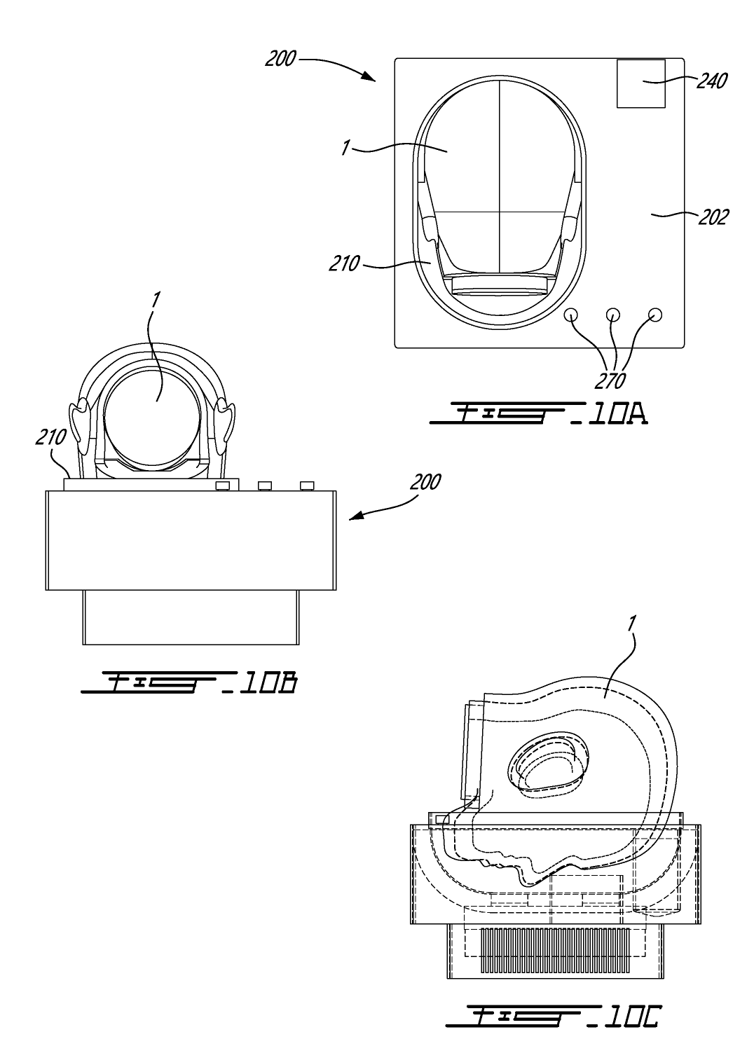

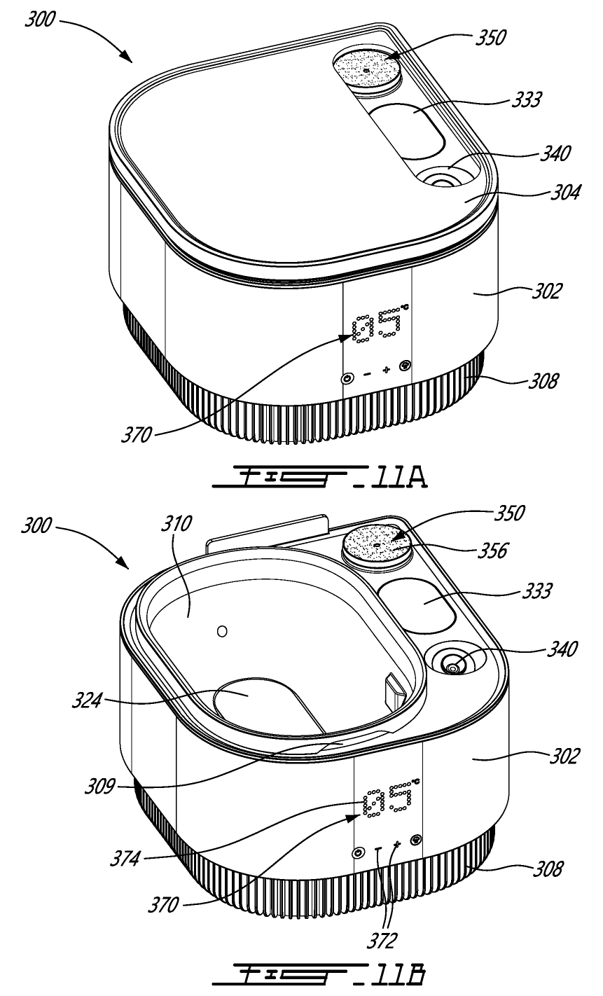

Referring now to FIGS. 11A and 11B, a personal thermal skin treatment device 300 according to another implementation is shown. The personal thermal skin treatment device 300 includes a housing 302 with a cover 304 with an aperture 305 that is removably coupled to a top side of the housing 302. The housing 302 supports a basin 310 on an external side thereof and contains a cooling module (not shown) and a heating module (not shown) on an internal side thereof. The external side of the housing 302 also includes a removable cover 333 for a water tank of the heating module, a nozzle 340 for expelling steam generated by the heating module, a diffuser 350 with a diffusing stone 356 for aromatherapy, and a control panel 370. The control panel 370 includes a user display 372 that shows the temperature and actuation buttons 374, for example, for the machine, the cooling module, and/or the heating module (i.e., turn on and off), set a desired temperature or temperature range for the cooling module and/or heating module, set or adjust a timer to maintain the liquid in the basin 310 at the desired temperature or expel steam from the nozzle 340 for a predetermined time. The housing 302 further contains a vent 308 on a bottom side thereof to assist with the dissipation of undesirable hot or warm air generated by the cooling module and/or heating module. The housing may be equipped with a light 338 located in close proximity to the nozzle 340 so as to illuminate the jet of steam from underneath.

In the exemplary implementation, the basin 310 is removably coupled to the housing 302 and includes a depression 309 to facilitate easier removal of the basin 310 from the housing 302. The basin 310 further includes plates 324 of the cooling module that are integrally formed on a bottom side of the basin 310 (i.e., the bottom part of the basin 310 is made of a material that facilitates thermal transfer from the cooling module to the basin 310). This implementation differs from the personal thermal skin treatment device 100 in that the cooling module includes a single plate 324 that is integrally formed with the basin 310.

Referring now to FIG. 12, a personal thermal skin treatment device 400 according to another implementation is shown. The personal thermal skin treatment device 400 includes a housing 402 with a basin 410 integrally formed as part of the housing 402, a removable cover 433 for a water tank of the heating module, a nozzle 440, and a diffuser 450. The personal thermal skin treatment device 400 further includes a cover 404 hingedly coupled to the housing 402 and configured to cover the basin 410 and a second cover 407 hingedly coupled to the housing 402 and configured to cover the removable cover 433, the nozzle 440, the diffuser 450 and the optional light 438. In the exemplary implementation, a top side of the integrally formed basin 410 is flush with the top surface of the housing 402 and therefore, the cover 404 does not include a groove or aperture to receive the basin 410. A digital control panel 470 is also provided to control all functions of the personal thermal skin treatment device 400.

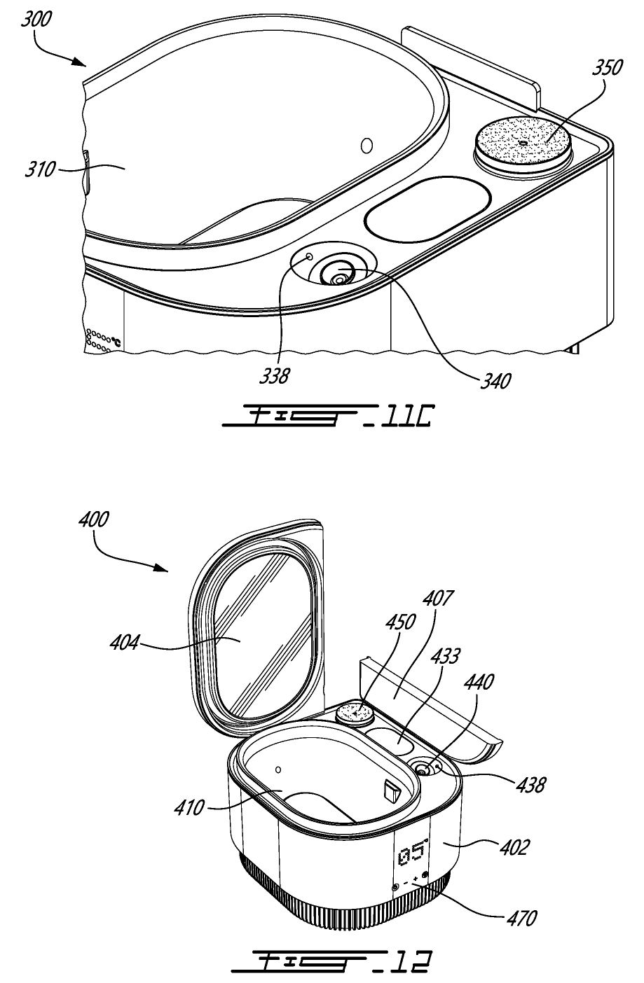

Referring now to FIG. 13, a personal thermal skin treatment device 500 according to another implementation is shown. The personal thermal skin treatment device 500 includes a housing 502 with a cover 504 removably coupled thereto. A basin 510 on the housing 502 includes a flange 562 and a lip 564 that protrudes from the housing 502. The cover 504 includes a groove 568 configured to receive the flange 562 and lip 564 on the basin 510 when the cover 504 is coupled to the basin 510 and housing 502.

In this implementation, the housing 502 does not include a nozzle. In such implementations, the housing 502 can contain only a cooling module or the housing 502 can contain a cooling module and a heating module that are both configured to cool or heat, respectively, a thermally conductive plate 524 formed in a bottom side of the basin 510. In other implementations, the nozzle can be implemented within the basin 510 such that the heating module is configured to expel steam out the nozzle into the basin 510.

Referring now to FIG. 14, a personal thermal skin treatment device 600 according to another implementation is shown. The personal thermal skin treatment device 600 includes a housing 602 with a basin 610, diffuser 650, and control panel 670. The personal thermal skin treatment device 600 includes a cooling module (not shown) that cools the basin 610, which in turn cools a liquid contained in the basin 610. In the exemplary implementation, the basin 610 is formed of a high thermal conductivity material that allows the cooling effect of the cooling module to be transferred easily to the liquid in the basin 610. In such implementations, the cooling module can abut the bottom and/or sides of the basin 610 to directly cool the basin 610. The cooling module can directly abut the basin 610 or indirectly contact the basin 610 through spacers.

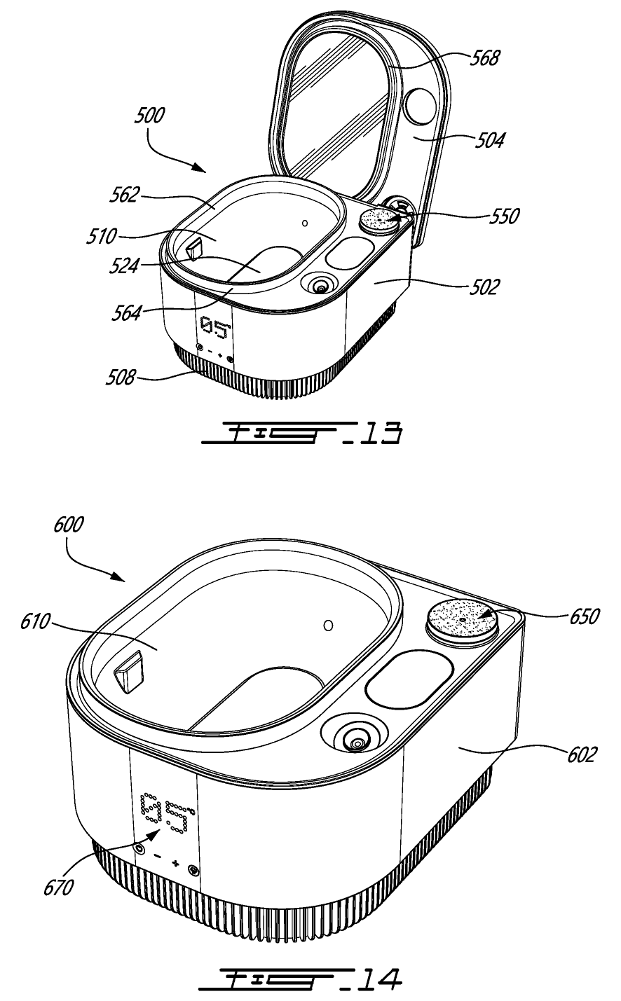

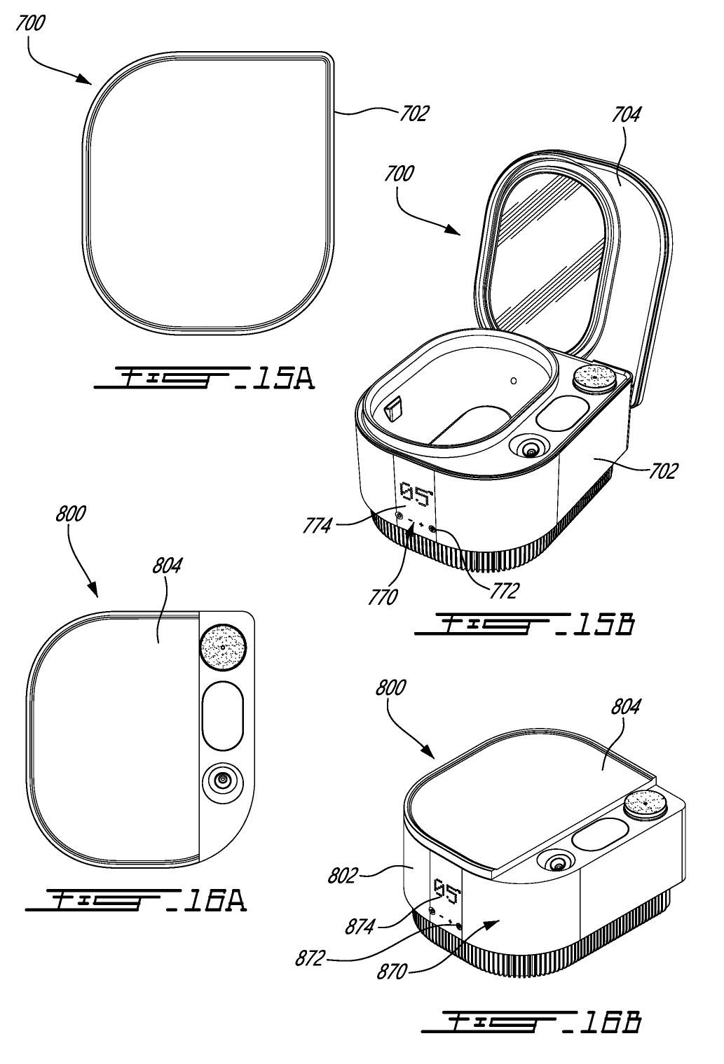

Referring now to FIGS. 15A and 15B, a personal thermal skin treatment device 700 according to another implementation is shown. The personal thermal skin treatment device 700 includes a housing 702 with a cover 704 hingedly attached to the housing 702. The housing 702 includes a control panel 770 having a user display 774 that shows the temperature and actuation buttons 772.

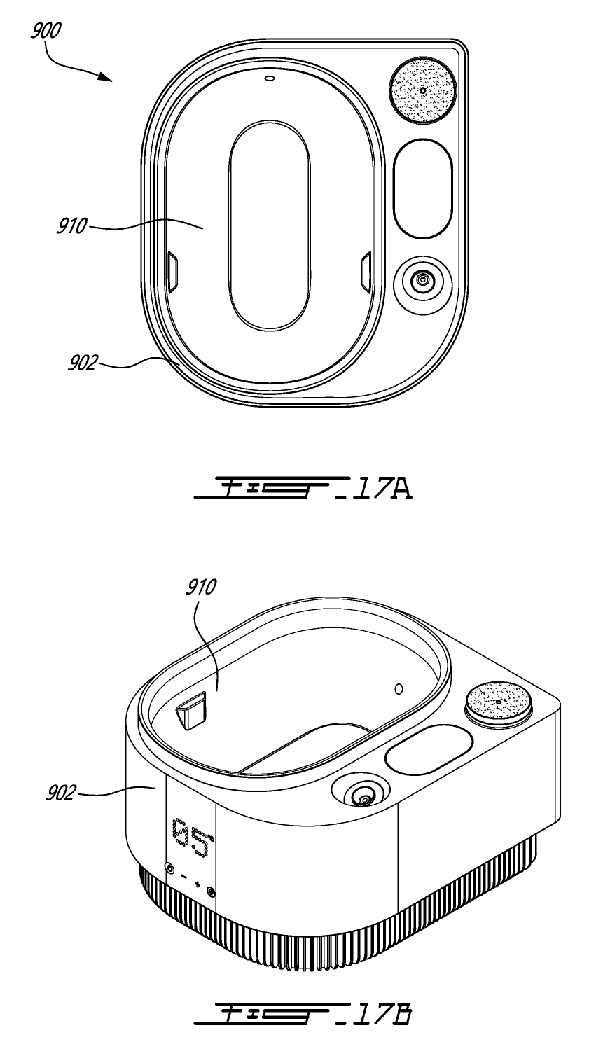

Referring now to FIGS. 16A and 16B, a personal thermal skin treatment device 800 according to another implementation is shown. The personal thermal skin treatment device 800 includes a housing 802 with a cover 804 coupled thereto. The housing includes a control panel 870 having a user display 874 that shows the temperature and an actuation buttons 872.

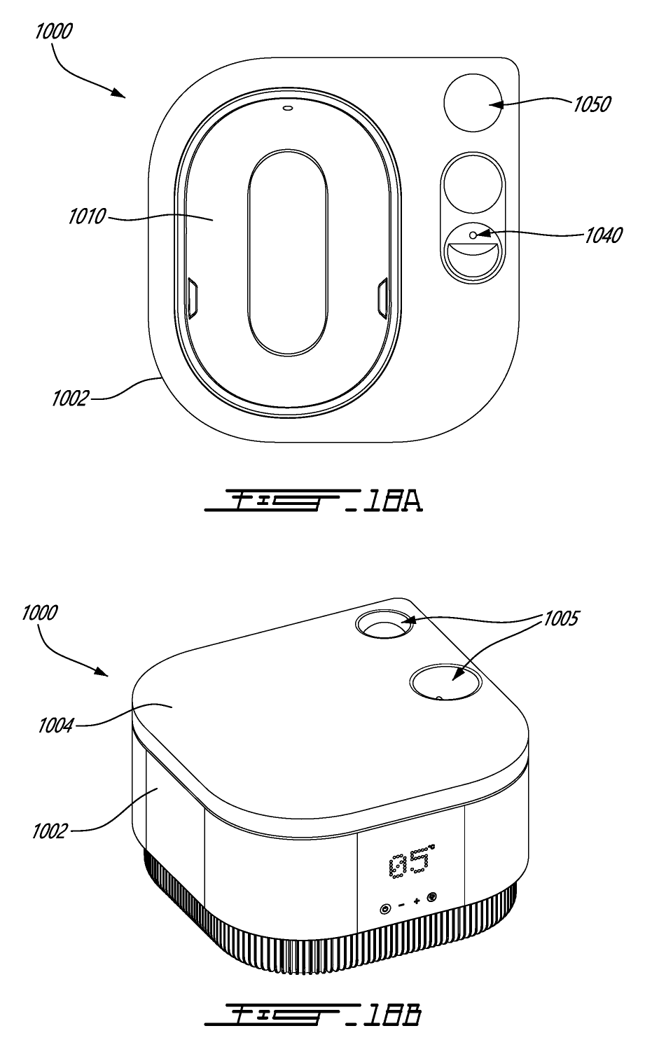

Referring now to FIGS. 17A and 17B, a personal thermal skin treatment device 900 according to another implementation is shown. The personal thermal skin treatment device 900 includes a housing 902 with an integrally formed basin 910.

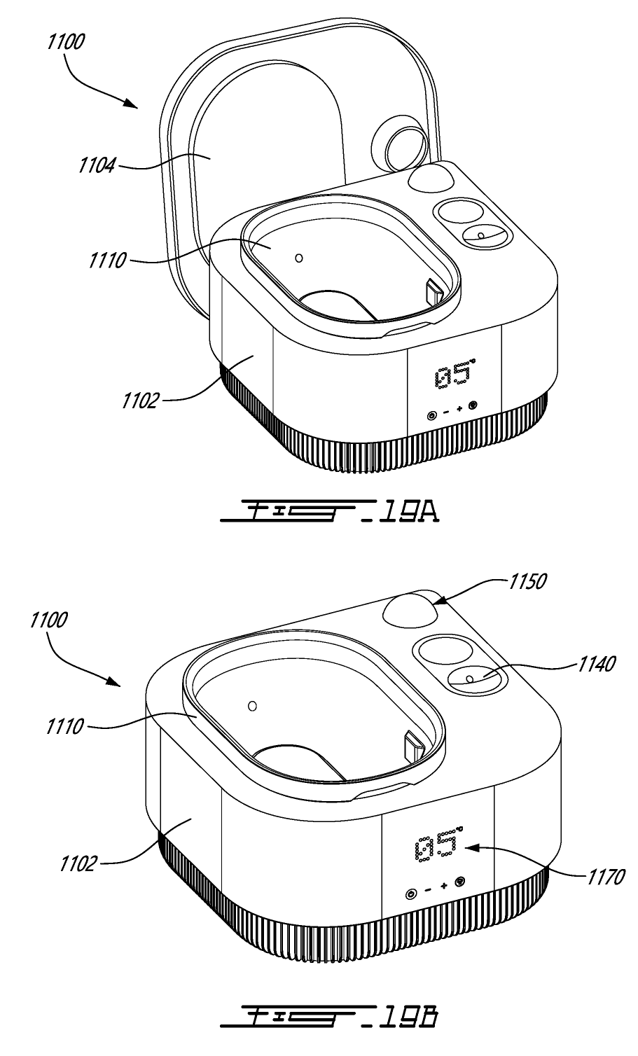

Referring now to FIGS. 18A and 18B, a personal thermal skin treatment device 1000 according to another implementation is shown. The personal thermal skin treatment device 1000 includes a housing 1002 with a cover 1004 having two apertures 1005. The housing 1002 supports a basin 1010, a nozzle 1040, and a diffuser 1050. The apertures 1005 are configured to align with the diffuser 1050 and the nozzle 1040 when the cover 1004 is coupled to the housing 1002.

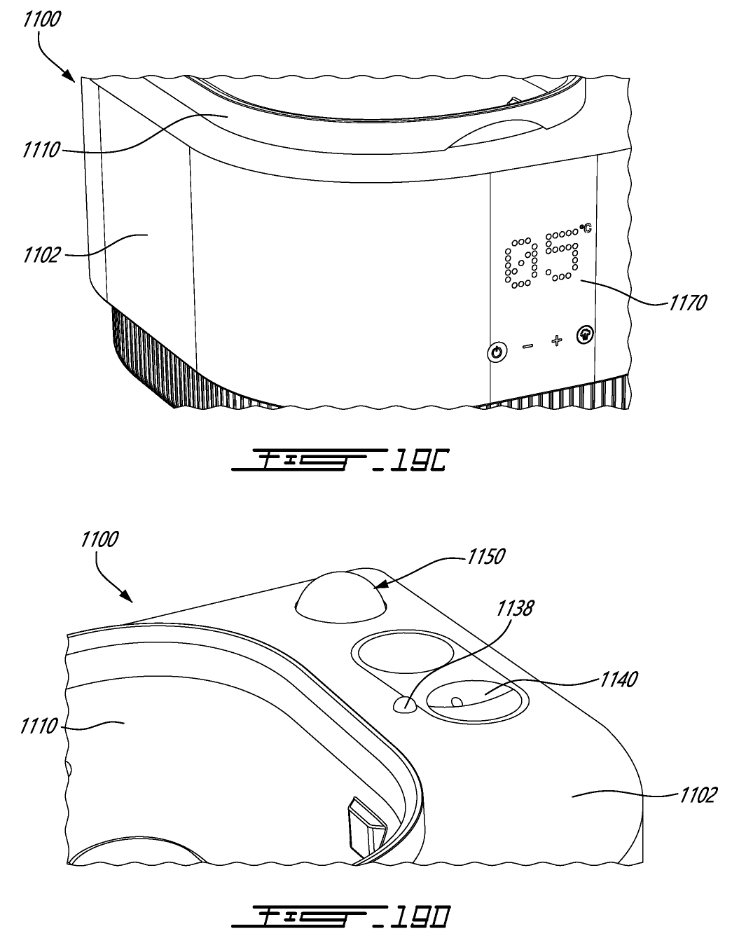

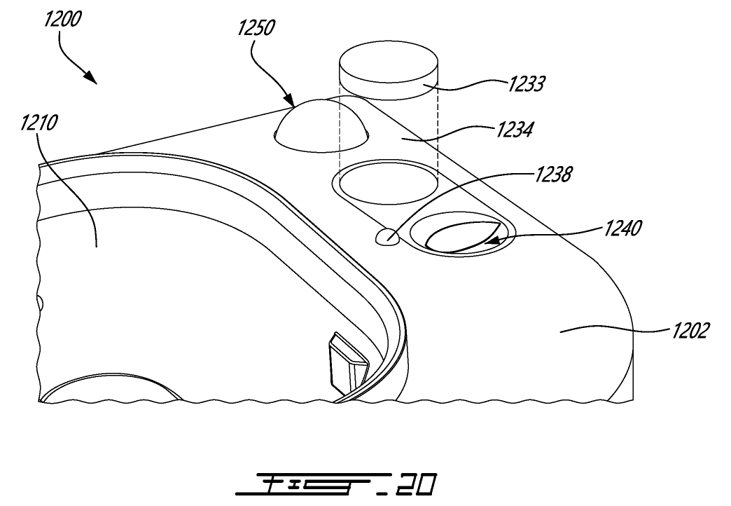

Referring now to FIGS. 19A-19E, a personal thermal skin treatment device 1100 according to another implementation is shown. The personal thermal skin treatment device 1100 includes a housing 1102 with a cover 1104 removably coupled thereto. The housing 1102 supports a basin 1110 that is removably coupled to the housing 1102, a nozzle 1140, a diffuser 1150, and a control panel 1170. The housing 1102 may be equipped with a light 1138 located in close proximity to the nozzle 1140 so as to illuminate the jet of steam emanating from the nozzle 1140 from underneath.

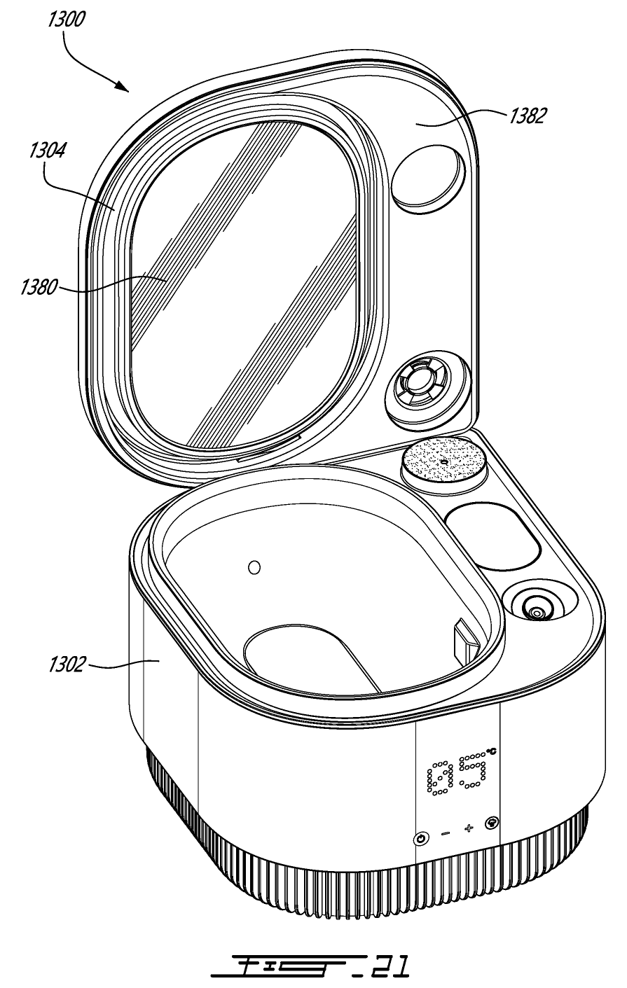

Referring now to FIG. 20, a personal thermal skin treatment device 1200 according to another implementation is shown. The personal thermal skin treatment device 1200 includes a housing 1202 supporting a basin 1210, a nozzle 1240 for expelling steam, and a diffuser 1250. The housing 1202 further contains a cooling module (not shown) configured to cool a liquid in the basin 1210 and a heating module (not shown) configured to heat a heatable liquid and expel steam through the nozzle 1240. The housing 1202 may be equipped with a light 1238 located in close proximity to the nozzle 1240 so as to illuminate from underneath the jet of steam emanating from the nozzle 1240.

The heating module includes a heating element in communication (such as fluid communication or heat transferable communication, whether by conduction, convention, an/or radiation) with a fluid tank 1234 configured to hold a heatable liquid. The fluid tank 1234 is in fluid communication with the nozzle 1240, such that when the heating element heats the heatable liquid in the fluid tank 1234, steam is generated and expelled from the nozzle 1240. In the exemplary implementation, the fluid tank 1234 includes a removable cover 1233 that the user can remove to fill the fluid tank 1234 with the heatable liquid. When the heating module is actuated, the fluid tank 1234 releases the heatable liquid to be in communication with the heating element, such that the heating element generates the steam, which is then expelled from the nozzle 1240.

Referring now to FIG. 21, a personal thermal skin treatment device 1300 according to another implementation is shown. The personal thermal skin treatment device 1300 includes a housing 1302 with a cover 1304 hingedly attached to the housing 1302. The cover 1304 includes a mirror 1380 on its inner face 1382. The mirror 1380 may be a magnifying mirror, such as a 10x mirror. The mirror 1380 can also be implemented on any other embodiments of the personal thermal skin treatment device having a cover, such as, in a non-limiting example, the personal thermal skin treatment devices 100, 300, 400, 500, 700, 800, 1000, 1100 and 1200.

Several alternative implementations and examples have been described and illustrated herein. The implementations of the technology described above are intended to be exemplary only. A person of ordinary skill in the art would appreciate the features of the individual implementations, and the possible combinations and variations of the components. A person of ordinary skill in the art would further appreciate that any of the implementations could be provided in any combination with the other implementations disclosed herein. It is understood that the technology may be embodied in other specific forms without departing from the central characteristics thereof. The present implementations and examples, therefore, are to be considered in all respects as illustrative and not restrictive, and the technology is not to be limited to the details given herein. Accordingly, while the specific implementations have been illustrated and described, numerous modifications come to mind.

As used herein, “substantially”, “approximately”, and/or “about” means an acceptable variation according to conventional standards, otherwise at most a 5% to 10% variation from an indicated effect or value.

What is claimed is:

1. A personal thermal skin treatment device comprising:
a housing;
a basin for receiving a liquid to submerge a body part of a user;
a cooling module thermally coupled to the basin and configured to cool the liquid in the basin;
a heating module for producing heat; and
thermal insulation, the thermal insulation being located between the heating module and the basin, between the heating module and the cooling module or both,
wherein the basin, the cooling module, the heating module and the thermal insulation are located at least partially within the housing.

2. The personal thermal skin treatment device of claim 1, wherein the thermal insulation is at least one of:
an insulation barrier to create compartments to at least partially contain the cooling module or the heating module;
a heat insulation panel positioned between the heating module and the basin; or
a heat insulation panel positioned between the heating module and the cooling module.

3. The personal thermal skin treatment device of claim 2, wherein the basin is removably insertable in the housing.

4. The personal thermal skin treatment device of claim 1, further comprising a nozzle and a fluid tank for containing a liquid, wherein the fluid tank is connected to the nozzle.

5. The personal thermal skin treatment device of claim 4, further comprising a light proximate the nozzle for lighting the steam directed by the nozzle.

6. The personal thermal skin treatment device of claim 4, wherein the heating module is operable to produce steam by evaporating the liquid in the fluid tank using the heat, and the nozzle being operable to direct the steam produced by the heating module outside of the housing.

7. The personal thermal skin treatment device of claim 6, wherein the heating module further comprises a heating element thermally coupled to the fluid tank, the heat being at least partially produced by the heating element.

8. The personal thermal skin treatment device of claim 1, wherein the cooling module and the heating module are respectively a cooling side and a heating side of a thermoelectric module, the cooling side being thermally coupled to a thermally conductive portion of the basin to cool the liquid in the basin, the heating side being operable to produce the heat.

9. The personal thermal skin treatment device of claim 8, further comprising a spacer, wherein the thermal insulation is between the basin and the thermoelectric module to thermally insulate the basin from the heating side of the thermoelectric module and wherein the spacer extends through the thermal insulation and abuts the basin to thermally couple the cooling side of the thermoelectric module to the basin through the thermal insulation.

10. The personal thermal skin treatment device of claim 9, further comprising an inner basin wall supporting the basin and an outer basin wall spaced apart from the inner basin wall, wherein the thermal insulation is between the inner basin wall and the outer basin wall.

11. The personal thermal skin treatment device of claim 10, wherein the thermal insulation is an air or vacuum gap between the inner basin wall and the outer basin wall.

12. The personal thermal skin treatment device of claim 10, wherein the inner basin wall and the outer basin wall include an aperture to receive the spacer for thermally coupling the cooling side of the thermoelectric module to the basin through the thermal insulation.

13. The personal thermal skin treatment device of claim 12, wherein the aperture comprises walls separating the spacer from the thermal insulation.

14. The personal thermal skin treatment device of claim 8, further comprising a heat sink thermally coupled to the heating side of the thermoelectric module.

15. The personal thermal skin treatment device of claim 14, further comprising an insulation layer between the cooling side of the thermoelectric module and the heat sink to thermally insulate the cooling side and the basin from the heat sink.

16. The personal thermal skin treatment device of claims claim 1, further comprising a diffuser configured to disperse one or more essential oils, the diffuser having a diffuser nozzle or a diffuser stone.

17. The personal thermal skin treatment device of claim 1, further comprising an ionizer in fluid communication with the basin, the ionizer being operative to ionize the liquid in the basin.

18. A personal thermal skin treatment device comprising:
a housing;
a basin for receiving a liquid to submerge a body part of a user;
a cooling module having a cooling side and a heating side, the cooling side being thermally coupled to the basin for cooling the liquid in the basin;
a heating module for producing steam;
a nozzle to direct the steam produced by the heating module; and
thermal insulation, the thermal insulation being in at least one of the following locations:
between the basin and the heating side of the cooling module;
between the basin and the heating module;
between the cooling side of the cooling module and the heating side of the cooling module; and
between the cooling side of the cooling module and the heating module,
wherein the basin, the cooling module, the heating module, the thermal insulation and the nozzle are located at least partially within the housing.

19. The personal thermal skin treatment device of claim 18, wherein the cooling side and the heating side are the respective cooling side and heating side of a thermoelectric module.

20. The personal thermal skin treatment device of claim 18, further comprising a light proximate the nozzle for lighting the steam directed by the nozzle.
